(19) United States
(12) Patent Application Publication
(10) Pub. No.: US 2025/0133814 A1
(43) Pub. Date: Apr. 24, 2025

(54) MINIATURIZED IMPLANTABLE MEDICAL DEVICES AND ASSOCIATED FABRICATION TECHNIQUES

(71) Applicant: University of Florida Research Foundation, Incoprated, Gainesville, FL (US)

(72) Inventors: Adam Khalifa, Gainesville, FL (US); Shiva Sai Yeshala, Gainesville, FL (US)

(21) Appl. No.: 18/886,615

(22) Filed: Sep. 16, 2024

Related U.S. Application Data
(60) Provisional application No. 63/591,977, filed on Oct. 20, 2023.

(51) Int. Cl. H01L 21/8238; A61N 1/05; A61N 1/36; H01L 21/02; H01L 21/768; H01L 23/66

(52) U.S. Cl. CPC H10D 84/038; A61N 1/0529; A61N 1/3605; H01L 21/02068; H01L 21/76862; H01L 23/66; H10D 84/0186

(57) ABSTRACT

A fabrication technique for a miniaturized implantable medical device is described. The technique includes fabricating microelectrodes on the sidewalls of a complimentary metal-oxide-semiconductor (CMOS) chip. By employing atomic layer deposition (ALD) and plasma-focused ion beam (FIB) milling, the technique eliminates the necessity for off-chip components, thereby reducing the device's overall size and enhancing its reliability. This novel approach provides a fully integrated, single-package solution for wireless, battery-free neural interfacing, offering advantages over traditional methods that rely on complex packaging, assembly and external components.

### CROSS-REFERENCE TO RELATED APPLICATIONS

This application claims priority to U.S. Provisional Application Ser. No. 63/591,977, filed on Oct. 20, 2023, the contents of which are hereby incorporated by reference in their entirety.

### FIELD OF THE DISCLOSURE

The present application relates to implantable medical devices (IMDs), and in particular, systems, methods, and devices associated with the miniaturization of wireless implantable medical devices for neural interfaces.

### BACKGROUND

Implantable Medical Devices (IMDs) may be utilized for a variety of applications, notably for neural network modulation and brain activity monitoring. Cutting-edge developments in complementary metal-oxide-semiconductor (CMOS) technology have enabled the creation of highly miniaturized, low-power integrated circuits (ICs). Such ICs, in conjunction with innovative micro/nanofabrication techniques and advanced wireless connections, may enable the eradication of batteries and the development of IMDs that integrate multiple components for interfacing with the nervous system. These wireless, unattached, IMDs, commonly known as wireless motes, dust, or microdevices, represent the vanguard of electrode technology in neural interfaces. However, the miniaturization of IMDs presents significant challenges. Conventional IMDs may utilize off-chip components, however, the overall assembly and packaging design of the system may be pivotal in maintaining the small dimensions of the IMD. Contemporary wirelessly powered neural interfaces may consist of an assembly of various components. The techniques used to connect these components, such as flip chip bonding, wire bonding, and feedthroughs, may inadvertently increase the volume of an IMD and compromise its reliability (e.g., due to excessive interfaces and connections). Notably, the integration of components like the off-chip coil, electrodes, and surface mount devices may increase the volume of the IMD and decrease reliability.

In a move away from the utilization of off-chip components, researchers are exploring process flows that enable the integration of electrodes directly into an application-specific integrated circuit (ASIC), paving the way for more efficient, reliable, and compact IMDs. In one example of such an IMD, one or more microelectrodes for the IMD may be mounted onto the top surface of an ASIC. However, mounting one or more microelectrodes onto the top surface of the ASIC may lead to a substantial increase in surface area of the top surface, and consequently, a larger IMD. Furthermore, the current techniques to incorporate electrodes onto the ASIC, are complex and time-consuming.

### BRIEF SUMMARY

The invention discloses a method for miniaturizing neural implantable medical devices (IMDs) by fabricating microelectrodes on the sidewalls of a complimentary metal-oxide-semiconductor (CMOS) chip. By employing atomic layer deposition (ALD) and plasma-focused ion beam (FIB) milling, the invention eliminates the necessity for off-chip components, thereby reducing the device's overall size and enhancing its reliability. This novel approach provides a fully integrated, single-package solution for wireless, battery-free neural interfacing, offering advantages over traditional methods that rely on complex packaging, assembly, and external components.

A device is provided in accordance with an example embodiment. The device comprises a substrate comprising a first material, wherein a top surface of the substrate extends along a width dimension and a depth dimension; a region comprising a second material formed in contact with the substrate, wherein a first sidewall of the region and a second sidewall of the region extend along a height dimension and the depth dimension, the first sidewall of the region parallel to the second sidewall of the region; circuitry formed in the region comprising the second material; a first electrode coupled with the circuitry, wherein a portion of the first sidewall of the region exposes a sidewall of the first electrode; and a second electrode coupled with the circuitry, wherein a portion of the second sidewall of the region exposes a sidewall of the second electrode.

According to one embodiment, the region comprises a coil, the coil coupled with (e.g., connected to) the circuitry. According to one embodiment, the device further comprises: a first layer comprising a third material, the first layer formed in contact with the sidewall of the first electrode; and a second layer comprising the third material, the second layer formed in contact with the sidewall of the second electrode. According to one embodiment, the device further comprises: a third layer comprising a fourth material, the third layer formed in contact with the first layer and the first sidewall of the region; and a fourth layer comprising the fourth material, the fourth layer formed in contact with the second layer and the second sidewall of the region. According to one embodiment, a volume of the device is less than or equal to nine cubic microns.

Embodiments provided herein include a method of fabricating a device including: processing a substrate by at least one of dicing or machining the substrate; cleaning the substrate to remove debris; assembling the device; removing material to expose at least a portion of one or more electrodes of the device; and forming a material layer on the at least the portion of the one or more electrodes of the device. According to some embodiments, forming the material layer on the at least a portion of the one or more electrodes includes depositing a layer of platinum on the at least a portion of the one or more electrodes.

According to certain embodiments, depositing a layer of platinum on the at least a portion of the one or more electrodes includes using plasma focused ion beam deposition (PFID) to perform ion beam-induced deposition (IBID). According to some embodiments, removing material to expose at least a portion of one or more electrodes of the device includes performing one or more focused ion beam (FIB) milling operations on the device. The material removed in some embodiments includes material in regions aligned with exposed portions of the one or more electrodes. The removed portion of certain embodiments has a dimension of around 200 μm×4 μm.

According to some embodiments cleaning the substrate includes applying one or more solutions or liquids to remove the debris. The one or more solutions or liquids can include one or more of deionized water or isopropyl alcohol. According to some embodiments assembling the device includes assembling components of the device using a silver epoxy. According to certain embodiments assembling the device further includes encapsulating the device by forming a region that encapsulates the device.

### BRIEF DESCRIPTION OF DRA WINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

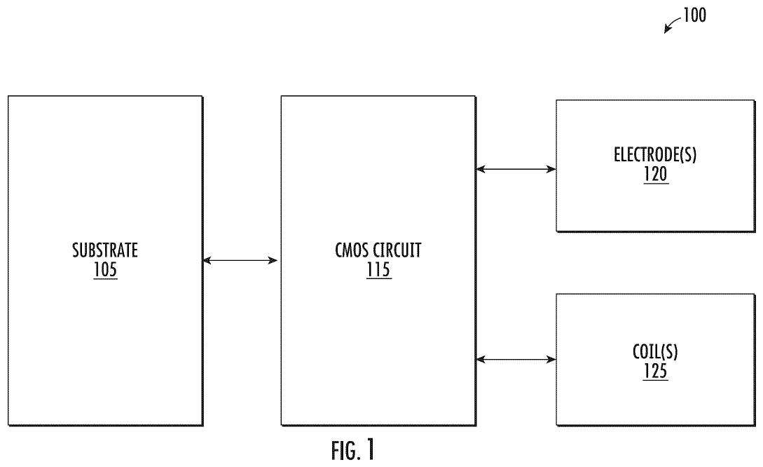

FIG. 1 illustrates an example of a system that supports miniaturized implantable medical devices (IMDs) and associated fabrication techniques according to an example embodiment of the present disclosure;

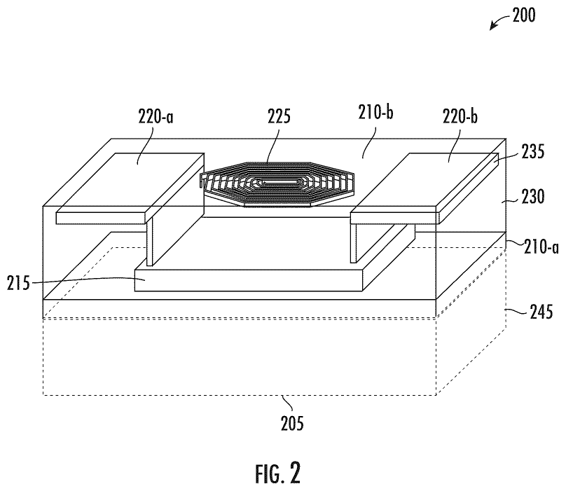

FIG. 2 is an example of a device that supports miniaturized IMDs and associated fabrication techniques according to an example embodiment of the present disclosure;

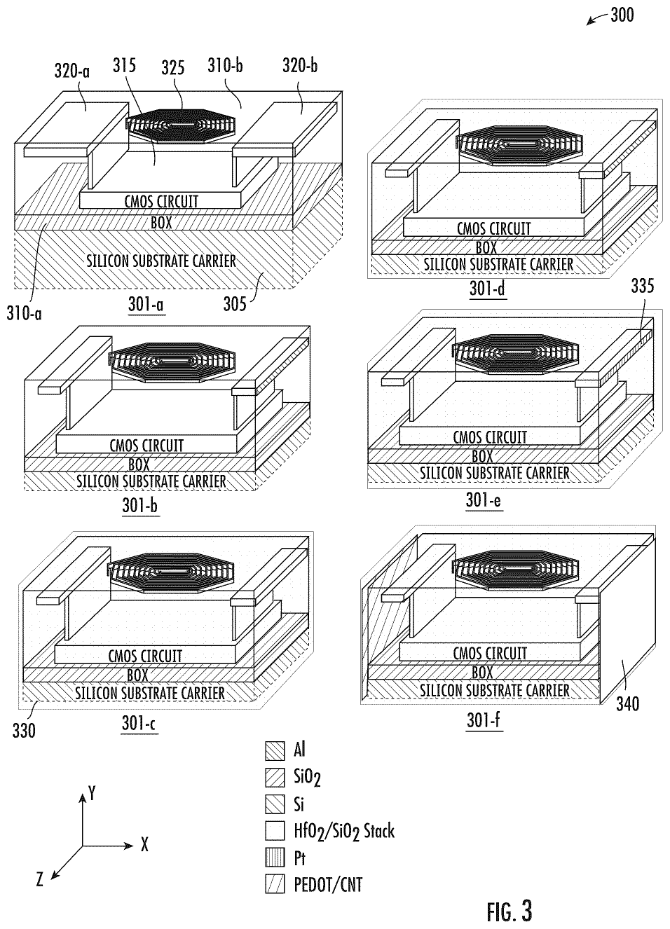

FIG. 3 is an example of a process flow that supports miniaturized IMDs and associated fabrication techniques according to an example embodiment of the present disclosure;

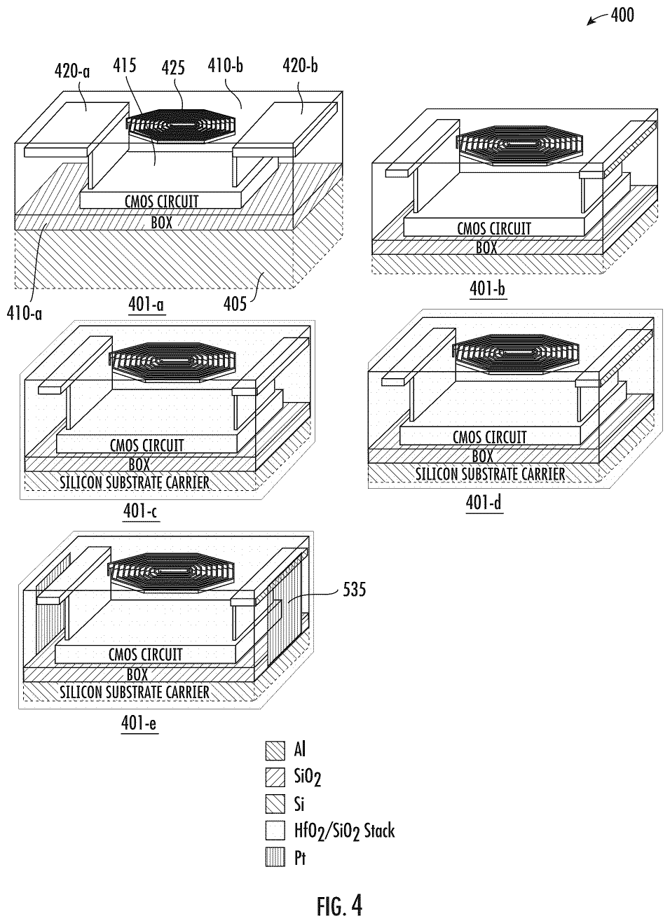

FIG. 4 is another example of a process flow that supports miniaturized IMDs and associated fabrication techniques according to an example embodiment of the present disclosure;

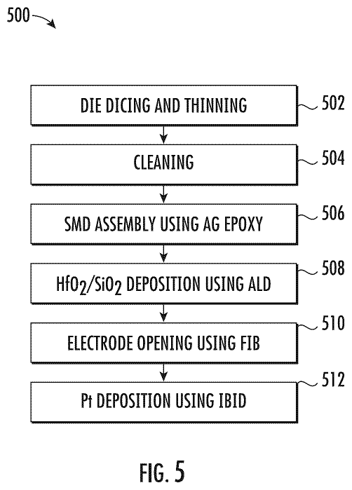

FIG. 5 is an example of a flowchart that supports miniaturized IMDs and associated fabrication techniques according to an example embodiment of the present disclosure; and

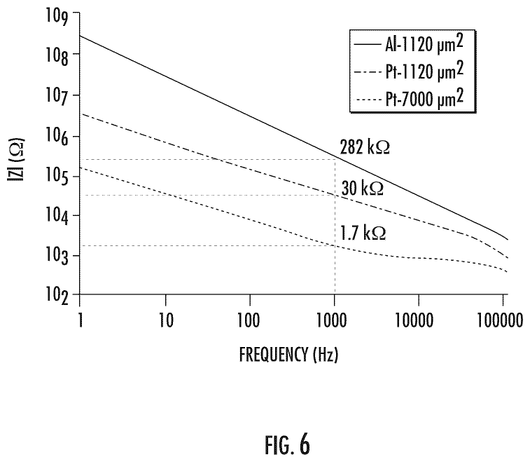

FIG. 6 is a chart illustrating impedance measurements for three electrode types according to an example embodiment of the present disclosure.

### DETAILED DESCRIPTION

Various embodiments of the present disclosure now will be described more fully hereinafter with reference to the accompanying drawings, in which some, but not all embodiments of the disclosure are shown. Indeed, the disclosure may be embodied in many different forms and should not be construed as limited to the embodiments set forth herein. Rather, these embodiments are provided so that this disclosure will satisfy applicable legal requirements. The term “or” is used herein in both the alternative and conjunctive sense, unless otherwise indicated. The terms “illustrative,” “example,” and “exemplary” are used to be examples with no indication of quality level. Like numbers refer to like elements throughout.

The present disclosure provides improvements to the technical field of implantable medical devices (IMDs). For example, the present disclosure includes techniques for fabricating electrodes on sidewalls of an IMD as opposed to the top or bottom surface of an IMD. Fabricating electrodes on sidewalls of an IMD may conserve space and may otherwise enable a reduction in the total volume of the IMD, which may reduce the invasiveness of implantation procedures. Reducing the size of the IMD may also allow for the implantation of multiple IMDs at different locations within the brain, which may increase the effectiveness of associated procedures. Additionally, fabricating electrodes on the sidewalls of an IMD may increase the separation between electrodes (e.g., may increase cathode anode separation), which may improve stimulation and recording abilities of the IMD. The fabrication techniques described herein may also provide reduced complexity when compared to other fabrication techniques for producing IMDs that have electrodes that are mounted on a top surface of the IMD.

FIG. 1 illustrates an example of a system 100 that supports miniaturized IMDs and associated fabrication techniques. The system 100 may include one or more components, which may be coupled (e.g., connected, electrically coupled, physically coupled, formed in contact with one another). For example, the system 100 may include a substrate 105, a complementary metal-oxide-semiconductor (CMOS) circuit 115, one or more electrodes 120, and one or more coils 125. In some cases, the system 100 may include one or more components that are not shown, such as a silicon dioxide (SiO₂) region, which may encapsulate the CMOS circuit 115, the electrodes 120, and the coils 125. As described herein, the system 100 may be an example of a an IMD, such as a neural interface or may otherwise be implanted into any other physiological tissue of a patient.

The substrate 105 may serve as a foundation for the system 100. In some cases, the CMOS circuit 115 may be formed in or on the substrate 105. The CMOS circuit 115 may include one or more components for controlling the system 100. The CMOS circuit 115 may be in communication with the one or more electrodes 120 and the one or more coils 125. For example, the CMOS circuit may transmit or receive one or more signals to or from the one or more electrodes 120. The one or more electrodes 120 may be in communication with (e.g., be in contact with) the physiological tissue of the patient. In some cases, the one or more coils 125 may be used to receive one or more signals from another device. The one or more signals may provide power for the system 100, which may be delivered to the CMOS circuit 115 via the one or more coils 125.

In some cases, the system 100 may be utilized to deliver or administer a treatment to a patient for a neurological condition. For example, the CMOS circuit 115 may transmit a signal to the one or more electrodes 120, which may result in one or more pulses being applied to one or more regions of the patient's brain. Applying the one or more pulses to the one or more regions of the patient's brain may result in alleviation of or treatment for the neurological condition. Although some examples described herein refer to the system 100 serving as a neural interface, the system may be utilized in other medical applications.

FIG. 2 illustrates an example of a device 200 that supports miniaturized IMDs and associated fabrication techniques. The device 200 may be an example of an IMD (e.g., an application-specific integrated circuit (ASIC) and may be formed using one or more processes as described herein. The device 200 may include one or more components, which may be examples of corresponding components described with reference to FIG. 1. The one or more components may include a region 205 (e.g., a first layer, a substrate 105, a silicon substrate carrier), one or more regions 210 (e.g., one or more second layers), a region 215 (e.g., one or more third layers, circuitry, a CMOS circuit 115), one or more regions 220 (e.g., one or more fourth layers, one or more electrodes 120), and one or more regions 225 (e.g., one or more fifth layers, one or more coils 125). As described herein, FIG. 2 may illustrate as isometric view of the device 200, rotated such that one or more sidewalls of the device 200 may be viewed. As shown, the device 200 may include a sidewall 230 of the regions 210, a sidewall 235 of the region 220-b, and a sidewall 245 of the region 205. Although not shown in FIG. 2, the device 200 may additionally or alternatively include one or more sixth layers (e.g., one or more ink layers, one or more electrodes), which may be formed on (e.g., in contact with) the sidewall 230, the sidewall 235, and the sidewall 245.

The device 200 may be described with reference to a coordinate system, as shown. The coordinate system may include or otherwise indicate one or more dimensions or directions. For example, the coordinate system may include an x-direction (e.g., a first direction, a width dimension), a y-direction (e.g., a second direction, a height dimension), and a z-direction (e.g., a third direction, a depth dimension). As shown, the x-direction may extend along a width of the IMD, the y-direction may extend along a height of the IMD, and the z-direction may extend along a depth of the IMD. The IMD may have a top surface and a bottom surface that are elongated along the x-direction and the z-direction (e.g., parallel to an x-z plane). The IMD may have four sidewalls. Two of the four sidewalls may be elongated along the y-direction and the z-direction (e.g., parallel to a y-z plane). The other two of the four side sidewalls may be elongated along the y-direction and the x-direction (e.g., parallel to an x-y plane).

The device 200 may include the region 205, which may be formed of a first material (e.g., silicon). The region 205 may have a width (e.g., along the x-direction), a depth (e.g., along the z-direction), and a height (e.g., along the y-direction). The region 205 may have one or more surfaces. For example, the region 205 may have four sidewalls, a top surface, and a bottom surface. The region 205 may be an example of a substrate and may be coupled with the region 210-a.

The device 200 may include the one or more regions 210, which may be formed of a second material (e.g., silicon dioxide (SiO₂)). Although some examples described herein refer to the region 210-a and the region 210-b separately, the region 210-a and the region 210-b may, in some examples, comprise a single, continuous region. The region 210-a and the region 210-b may each have a width (e.g., along the x-direction), a depth (e.g., along the z-direction), and a height (e.g., along the y-direction). The region 210-a and the region 210-b may each have one or more surfaces, which may include four sidewalls, a top surface, and a bottom surface. In some examples, the region 210-b may be an example of an insulation region, which may electrically insulate components of the device 200 from one another or from other objects (not shown). For example, the region 210-b may insulate the region 225 from the regions 220. In some examples, the region 210-a may be an example of a buried oxide (BOX) layer.

The device 200 may include the region 215, which may be formed of one or more materials as described herein. The region 215 may have a width (e.g., along the x-direction), a depth (e.g., along the z-direction), and a height (e.g., along the y-direction). The region 215 may have one or more surfaces, which may include four sidewalls, a top surface, and a bottom surface. The region 215 may be an example of circuitry, such as a CMOS circuit or any other type of integrated circuit (IC). In some cases, the region 215 or one or more portions of the region 215 may be formed in contact with the region 210-a. The region 215 may include a charge pump rectifier, a reference circuit, and a regulator.

The device 200 may include one or more regions 220 (e.g., the region 220-a, the region 220-b), which may be formed of a fifth material (e.g., aluminum) or a sixth material (e.g., zinc, copper, or a combination of both). Although some examples described herein refer to the one or more regions 220 being formed of aluminum, zinc, cooper, or a composite material, any conductive material may be used. The one or more regions 220 may each have a width (e.g., along the x-direction), a depth (e.g., along the z-direction), and a height (e.g., along the y-direction). The one or more regions 220 may each have one or more surfaces, which may include four sidewalls, a top surface, and a bottom surface. The sidewall 235 may be an illustrative example of one sidewall of the region 220-b. The one or more regions 220 may be examples of electrodes or portions of electrodes. In some cases, the one or more regions 220 may be coupled with the region 215 (e.g., via a respective coupling region 240). A coupling region 240 (e.g., a coupling region 240-a, a coupling region 240-b) may be formed of any material as described herein, such as a conductive material, among other examples. Additionally, or alternatively, the one or more regions 220 may be coupled with an implantation medium, such as a brain or a portion of a brain. In some cases, one or more signals may be received by or transmitted by the one or more regions 220. In some cases, the device 200 may utilize a pulsed power transmission scheme (e.g., an analog transmission scheme), which may result in stimulation for each radio frequency (RF) pulse transmitted via the one or more regions 220. Such a configuration may enable an amount of charge injected into an implantation medium (e.g., tissue), which is determined by both a stimulus pulse width and strength, to correspond directly to a width of an RF pulse.

The device 200 may include one or more regions 225 (e.g., one or more coils), which may be formed of a fifth material (e.g., aluminum) or a sixth material (e.g., zinc). A region 225 may have a width (e.g., along the x-direction), a depth (e.g., along the z-direction), and a height (e.g., along the y-direction). A region 225 may also have a length (e.g., a total length of the coil). A region 225 may have one or more surfaces. In some cases, the region 225 may power the IMD. For example, the region 225 may harvest electrical energy transmitted by another device. In some cases, the region 225 may be printed or otherwise included on a printed circuit board (PCB).

In some cases, the device 200 may include one or more sixth layers (e.g., one or more ink layers), which may be formed using a fourth material which can include deposited platinum, or in some embodiments (e.g., poly(3,4-ethylenedioxythiophene) polystyrene sulfonate (PEDOT: PSS), a carbon nanotube (CNT) material). In some cases, the one or more sixth layers may be examples of or may otherwise be referred to as surfaces or layers, however, the one or more sixth layers may each have a width (e.g., along the x-direction), a depth (e.g., along the z-direction), and a height (e.g., along the y-direction). The one or more sixth layers may each have one or more surfaces, which may include four sidewalls, a top surface, and a bottom surface. The one or more sixth layers may be examples of electrodes or portions of electrodes (e.g., conductive ink regions coupled with electrodes). In some cases, the one or more sixth layers may be coupled with the region 215 via a respective region 220. Additionally, or alternatively, the one or more sixth layers may be coupled with an implantation medium, such as a brain or a portion of a brain. In some cases, one or more signals may be received by one or more of the sixth layers and conveyed to the region 215 via an electrical coupling.

As described herein, one or more electrical signals from an implantation medium (e.g., a brain) may be transmitted through a sixth layer and a region 220. Thus, respective sixth layers and regions 220 may be conductive and may thus be referred to separately as electrodes or in combination as a single electrode. For example, a sixth layer coupled with the region 220-b may be a first portion of an electrode and the region 220-b may be a second portion of the electrode. Additionally, the term “sidewall” may refer to any surface parallel to a y-z plane or an x-y plane. For example, the device 200 may have four sidewalls. It should also be noted that the terms “device 200,” “IMD,” “system,” “chip,” “implantable device,” “microdevice,” “microelectronic device,” and “ASIC” may be used interchangeably. Additionally, the region 215 may be referred to individually as an ASIC.

FIG. 3 illustrates an example of a process flow 300 that supports miniaturized IMDs and associated fabrication techniques. The process flow 300 may illustrate various states of an IMD during the process flow 300 (e.g., during a manufacturing process). The IMD may be formed using a variety of materials. The materials may include silicon (e.g., a first material), SiO₂ (e.g., a second material), platinum (e.g., a third material), PEDOT: PSS or a CNT material (e.g., a fourth material), aluminum (e.g., a fifth material), zinc (e.g., a sixth material), and hafnium oxide (HfO₂) (e.g., a seventh material). In some cases, the IMD may be formed using one or more composite materials (e.g., one or more combinations of the materials described herein). For example, an eighth material including HfO₂ and SiO₂ may be used (e.g., a transparent multilayer of HfO₂ and SiO₂).

The IMD shown in the process flow 300 may include one or more components, which may be examples of corresponding components described with reference to FIGS. 1 and 2. For example, the IMD may include a substrate 305 (e.g., a substrate 105, a region 205), one or more SiO₂ regions 310 (e.g., the one or more regions 210), circuitry 315 (e.g., the CMOS circuit 115, the region 215), one or more electrodes 320 (e.g., one or more electrodes 120, one or more regions 220), one or more coils 325 (e.g., one or more coils 125, one or more regions 225), and ink 340 (e.g., the region 240), which may be examples of corresponding components described with reference to FIGS. 1 and 2.

The process flow 300 may be described with reference to a coordinate system, as shown. The coordinate system may include or otherwise indicate one or more dimensions or directions. For example, the coordinate system may include an x-direction (e.g., a first direction, a width dimension), a y-direction (e.g., a second direction, a height dimension), and a z-direction (e.g., a third direction, a depth dimension). As shown, the x-direction may extend along a width of the IMD, the y-direction may extend along a height of the IMD, and the z-direction may extend along a depth of the IMD. The IMD may have a top surface and a bottom surface that are elongated along the x-direction and the z-direction (e.g., parallel to an x-z plane). The IMD may have four sidewalls. Two of the four sidewalls may be elongated along the y-direction and the z-direction (e.g., parallel to a y-z plane). The other two of the four side sidewalls may be elongated along the y-direction and the x-direction (e.g., parallel to an x-y plane).

At 301-a, various components of the IMD may be formed or otherwise assembled. For example, the components of the IMD may be formed using silicon-on-insulator (SOI) technology. In some cases, circuitry 315 (e.g., an active silicon layer) may be formed in a SiO₂ region 310 (e.g., a SiO₂ layer, a BOX layer), where the SiO₂ region 310 itself is formed on a substrate 305 (e.g., a silicon carrier substrate). In some cases, one or more electrodes 320 may be formed in the SiO₂ region 310. Additionally, one or more conducting regions 240 may be formed in the SiO₂ region 310. The one or more electrodes 320 may be formed using aluminum, copper, or a combination of both. The one or more conducting regions 240 may be formed using aluminum, cooper, or a combination of both. In some cases, one or more coils 325 may be formed in the SiO₂ region 310. The one or more coils 325 may be formed using aluminum, copper, or a combination of both. In some cases, the operations described with reference to 301-a may be performed prior to one or more dicing operations.

At 301-b, one or more material removal operations may be performed, such as one or more dicing operations, which may result in the formation of one or more IMD dies (e.g., formation of the IMD). The one or more material removal operations may result in the formation of a 180 micron (μm)×180 μm IMD. In some cases, the dicing operation may include removing material from each sidewall of the IMD. In some cases, the one or more material removal operations may expose (e.g., reveal) the aluminum metallization (e.g., the one or more electrodes 320) on one or more sides of the IMD, which may function as both stimulation and recording electrodes. For example, the one or more dicing operations may expose sidewalls of the one or more electrodes 320.

In some cases, the techniques and associated IMD configurations described herein may eliminate that need for three dimensional electrodes (e.g., electrodes that protrude outwards, and extend beyond a surface of the IMD). For example, the IMD configurations described herein may be significantly smaller than other IMD configurations, which may enable the IMD (e.g., the entirety of the IMD system) to be implanted and positioned proximate to a target neuron. Such implantation of the entirety of the IMD system may enable a reduction in size of electrodes 320 (e.g., electrodes 320 may be aligned with and parallel to sidewalls of the IMD), which may reduce the size of and improve performance of the IMD. In some cases (e.g., subsequent to the one or more dicing operations), one or more additional material removal operations may be performed. For example, one or more thinning operations may be performed, which may further reduce a size of the IMD. The one or more additional material removal operations may include removing a portion of the substrate 305. For example, a height of the substrate 305 (e.g., a y-dimension of the substrate 305) may be reduced. Subsequent to the one or more additional material removal operations, a y-dimension of the IMD may be 40 μm. The one or more additional material removal operations may be performed using a polishing system (e.g., a MultiPrep® polishing system).

At 301-c, one or more material formation operations may be performed. The one or more material formation operations may include one or more deposition operations (e.g., one or more atomic layer deposition (ALD) operations). For example, one or more SiO₂ regions 310 may be formed on the substrate. The one or more SiO₂ regions 310 may encapsulate one or more components of the IMD. For example, the SiO₂ region 310-b may encapsulate the circuitry 315, the one or more electrodes 320, and the one or more coils 325. Although FIG. 3 shows one illustrative example of the SiO₂ region 310 being present at 301-a and at 301-b (e.g., being formed at or prior to 301-a), the SiO₂ region 310 may be formed at 301-c. In some other cases, the SiO₂ region 310 may be formed at any other phase or step of the process flow 300, such as at or prior to 301-a. In some cases, during an ALD operation, the IMD may be placed on a stainless-steel wire mesh with an opening of 276 μm for conformal deposition. In some cases, a region 330 may be formed at 301-c (e.g., an ultra-thin, transparent multilayer of SiO₂/HfO₂, an SiO₂/HfO₂ stack). The region 330 may encapsulate the IMD. In some cases, the region 330 may have a thickness that is less than or equal to 100 nanometers (nm). In some cases, the region 330 may be formed of SiO₂, HfO₂, or a composite including both SiO₂ and HfO₂.

At 301-d, one or more material removal operations may be performed. The one or more material removal operations may remove portions of the region 330. The one or more material removal operations may serve to integrate the one or more electrodes 320 (e.g., expose the one or more electrodes 320 via the removed portions of the region 330). In some cases, the one or more material removal operations may include focused ion beam (FIB) milling (e.g., plasma-focused ion beam (PFIB) milling), which may form openings in the region 330. The openings may expose sidewalls of the one or more electrodes 320 (e.g., aluminum may be exposed and etched). The openings may each have dimensions of 200 μm×4 μm. For example, a depth of an opening (e.g., along the z-direction) may be 200 μm and a height of the opening (e.g., along the y-direction) may be 4 μm.

At 301-e, one or more material formation operations may be performed. For example, an ion beam-induced deposition (IBID) operation may be performed. The IBD operation may include forming a layer 335 of platinum (Pt) on one or more electrodes 320 (e.g., on an exposed sidewall of one or more electrodes 320). In some cases, the layer 335 of platinum may have a thickness of 200 nm (e.g., a thickness along the x-direction for one exemplary electrode). Forming the layer 335 on the one or more electrodes 320 may prevent or reduce oxidation of the one or more electrodes 320. Although some examples described herein refer to forming a layer 335 of platinum on the one or more electrodes, other materials (e.g., gold) may be used.

At 301-f, one or more material formation operations may be performed. For example, a printing operation (e.g., an inkjet printing operation) may be performed to deposit an ink 340 on the one or more electrodes 320 (e.g., sidewalls of the one or more electrodes 320). As shown, the ink 340 may also be deposited on the region 330. The ink 340 may include one or more materials, such as a conductive polymer material (e.g., PEDOT: PSS), a CNT material, or both. The ink 340 may be an aqueous mixture. As one illustrative example, the ink may be Poly-ink HC®.

In some cases, the ink 340 may be passed through a glass fiber syringe filter with a pore size of 0.7 μm and degassed (e.g., for a duration, for one or more minutes) to remove trapped air bubbles before it is loaded into a cartridge. The ink 340 may be applied using an inkjet printer including one or more cartridges having drop volumes of ten picoliters (pLs). To guide the printing process, one or more images of the IMD may be recorded using an optical microscope. The one or more images may show one or more sidewalls of the IMD. Multiple cleaning cycles (e.g., of the IMD) and voltage adjustments (e.g., of the printer) may be performed until a trajectory of the droplet is stable. The IMD may be transferred with a tweezer to a carrier glass slide with a sidewall facing up. One or more parameters may be selected for the printing operation based on results of previous printing operations. For example, drop-to-drop spacing and size parameters as well as jet parameters (e.g., speed and frequency) may be selected based on the results of previous printing operations. A temperature of the cartridge and the device may be set to 28 degrees Celsius. The IMD may be baked on a hot plate and gradually cooled to room temperature. The die may then be flipped over with high-precision tweezers.

In some cases, any one or more of the operations described herein may be repeated for one or more other sidewalls of the IMD (e.g., for one or more other electrodes 320 of the IMD). Although some examples may refer to respective operations being performed for multiple electrodes 320 or multiple sidewalls of the IMD (e.g., simultaneously), the operations described herein may be repeated or otherwise performed sequentially for each electrode 320 of the IMD. For example, the operations performed at 301-a, at 301-b, at 301-c, and at 301-d may be performed once per IMD, and the operations performed at 301-e and 301-f may be performed once per electrode 320. The IMD may include any quantity of electrodes 320. In some cases, the IMD may include one electrode 320 per sidewall of the IMD. Although FIG. 3 shows one illustrative example of an IMD that includes two electrodes 320, the IMD may include additional electrodes 320 that are not shown, for clarity (e.g., the IMD may include an electrode 320 corresponding to the front sidewall and an electrode 320 correspond to the rear sidewall).

The operations described with reference to FIG. 3 may be referred to as being performed “at” various phases of the process flow 300. However, it should be noted that the various depictions of the IMD “at” various phases may represent the IMD after corresponding operations are performed. For example, the corresponding illustration of the IMD shown “at 301-b” may be an illustrative example of the IMD after operations described with reference to 301-b (e.g., the dicing operation, the thinning operation) are performed.

FIG. 4 illustrates another example of a process flow for forming a microdevice of an example embodiment described herein. The process flow 400, similar to that of process flow 300 of FIG. 3, may be described with reference to a coordinate system, as shown. As shown in FIG. 4, the x-direction may extend along a width of the IMD, the y-direction may extend along a height of the IMD, and the z-direction may extend along a depth of the IMD. The IMD may have a top surface and a bottom surface that are elongated along the x-direction and the z-direction (e.g., parallel to an x-z plane). The IMD may have four sidewalls. Two of the four sidewalls may be elongated along the y-direction and the z-direction (e.g., parallel to a y-z plane). The other two of the four side sidewalls may be elongated along the y-direction and the x-direction (e.g., parallel to an x-y plane).

At 401-a, various components of the IMD may be formed or otherwise assembled. For example, the components of the IMD may be formed using silicon-on-insulator (SOI) technology. In some cases, circuitry 415 (e.g., an active silicon layer) may be formed in a SiO₂ region 410 (e.g., a SiO₂ layer, a BOX layer), where the SiO₂ region 410 itself is formed on a substrate 405 (e.g., a silicon carrier substrate). In some cases, one or more electrodes 520 may be formed in the SiO₂ region 410. Additionally, one or more conducting regions may be formed in the SiO₂ region 410. The one or more electrodes 420 may be formed using aluminum, copper, or a combination of both. The one or more conducting regions may be formed using aluminum, cooper, or a combination of both. In some cases, one or more coils 425 may be formed in the SiO₂ region 410. The one or more coils 425 may be formed using aluminum, copper, or a combination of both. In some cases, the operations described with reference to 501-a may be performed prior to one or more dicing operations.

At 401-b, one or more material removal operations may be performed, such as one or more dicing operations, which may result in the formation of one or more IMD dies (e.g., formation of the IMD). The one or more material removal operations may result in the formation of a 180 micron (μm)×180 μm IMD. In some cases, the dicing operation may include removing material from each sidewall of the IMD. In some cases, the one or more material removal operations may expose (e.g., reveal) the aluminum metallization (e.g., the one or more electrodes 520) on one or more sides of the IMD, which may function as both stimulation and recording electrodes. For example, the one or more dicing operations may expose sidewalls of the one or more electrodes 420.

In some cases, the techniques and associated IMD configurations described herein may eliminate that need for three dimensional electrodes (e.g., electrodes that protrude outwards, and extend beyond a surface of the IMD). For example, the IMD configurations described herein may be significantly smaller than other IMD configurations, which may enable the IMD (e.g., the entirety of the IMD system) to be implanted and positioned proximate to a target neuron. Such implantation of the entirety of the IMD system may enable a reduction in size of electrodes 420 (e.g., electrodes 420 may be aligned with and parallel to sidewalls of the IMD), which may reduce the size of and improve performance of the IMD. In some cases (e.g., subsequent to the one or more dicing operations), one or more additional material removal operations may be performed. For example, one or more thinning operations may be performed, which may further reduce a size of the IMD. The one or more additional material removal operations may include removing a portion of the substrate 405. For example, a height of the substrate 405 (e.g., a y-dimension of the substrate 405) may be reduced. Subsequent to the one or more additional material removal operations, a y-dimension of the IMD may be 40 μm. The one or more additional material removal operations may be performed using a polishing system (e.g., a MultiPrep® polishing system).

At 401-c, one or more material formation operations may be performed. The one or more material formation operations may include one or more deposition operations (e.g., one or more atomic layer deposition (ALD) operations). For example, one or more SiO₂ regions 410 may be formed on the substrate. The one or more SiO₂ regions 410 may encapsulate one or more components of the IMD. For example, the SiO₂ region 410-b may encapsulate the circuitry 415, the one or more electrodes 420, and the one or more coils 425. Although FIG. 4 shows one illustrative example of the SiO₂ region 410 being present at 401-a and at 401-b (e.g., being formed at or prior to 401-a), the SiO₂ region 510 may be formed at 401-c. In some other cases, the SiO₂ region 310 may be formed at any other phase or step of the process flow 400, such as at or prior to 401-a. In some cases, during an ALD operation, the IMD may be placed on a stainless-steel wire mesh with an opening of 276 μm for conformal deposition. In some cases, a region may be formed at 401-c (e.g., an ultra-thin, transparent multilayer of SiO₂/HfO₂, an SiO₂/HfO₂ stack). The region may encapsulate the IMD. In some cases, the region may have a thickness that is less than or equal to 100 nanometers (nm). In some cases, the region may be formed of SiO₂, HfO₂, or a composite including both SiO₂ and HfO₂.

At 401-d, one or more material removal operations may be performed. The one or more material removal operations may serve to integrate the one or more electrodes 420 (e.g., expose the one or more electrodes 420 via the removed portions of the region). In some cases, the one or more material removal operations may include focused ion beam (FIB) milling (e.g., plasma-focused ion beam (PFIB) milling).

At 401-e, one or more material formation operations may be performed. For example, an ion beam-induced deposition (IBID) operation may be performed. The IBD operation may include forming a layer 435 of platinum (Pt) on one or more electrodes 420. In some cases, the layer 435 of platinum may have a thickness of 200 nm (e.g., a thickness along the x-direction for one exemplary electrode). Forming the layer 335 on the one or more electrodes 420 may prevent or reduce oxidation of the one or more electrodes 420. Although some examples described herein refer to forming a layer 435 of platinum on the one or more electrodes, other materials (e.g., gold) may be used.

FIG. 5 illustrates an example of a flowchart 500 that supports miniaturized IMDs and associated fabrication techniques. The flowchart 500 may include one or more operations that result in the formation of an IMD or one or more components of an IMD, as described herein. The operations of the flowchart 500 may be performed by one or more components of a manufacturing system. For example, the operations of the flowchart 500 may be performed by one or more machines specifically designed to perform the operations described herein. Although the flowchart 500 provides one illustrative example of an order of operations, the operations described herein may be performed in any order other than the order shown. In some cases, one or more operations may be omitted from the flowchart 500 (e.g., skipped). Additionally, or alternatively, one or more operations not shown in the flowchart may be performed.

At 502, one or more dicing and grinding operations may be performed on a substrate. For example, a dicing operation may reduce a size of an IMD by slicing or otherwise cutting sidewalls of the IMD. Additionally, or alternatively, a grinding operation may be performed to thin the substrate 405. For example, the grinding operation may reduce a height of the substrate 405 by thinning or removing a portion of the substrate 405.

At 504, a cleaning operation (e.g., a pre-cleaning operation) may be performed. The cleaning operation may remove debris from the IMD. The cleaning operation may include applying one or more solutions or liquids to the IMD. The one or more solutions or liquids may include deionized water (DI), isopropyl alcohol (IPA), or a combination thereof. In some cases, a gas may be utilized to remove debris from the IMD. For example, nitrogen gas may be utilized to clean the IMD. The cleaning operation may serve to prepare the IMD for one or more deposition or material formation operations to be performed after the cleaning operation.

At 506 a surface mounted device (SMD) may be assembled using, for example, a silver epoxy to mechanically fasten components to the substrate. At 508, one or more deposition operations may be performed. For example, one or more ALD operations may be performed. The one or more deposition operations may include depositing (e.g., forming) a region 430 that encapsulates the IMD (e.g., a multi-stack, a stack multiple materials, a stack of HfO₂ and SiO₂). As one illustrative example, the region 430 may include five layers of HfO₂ and five layers of SiO₂. Each layer may have a thickness of 10 nm. Accordingly, a total thickness of region 430 may be 100 nm. The layers of the multi-stack may alternate between HfO₂ and SiO₂.

At 510, one or more material removal operations may be performed. For one example, one or more FIB milling operations may be performed. The one or more material removal operations may include removing portions of the region 430. The removed portions of the region 430 may be aligned with the exposed portions (e.g., exposed sidewalls) of the one or more electrodes 420. In some cases, each of the removed portions of the region 430 may have dimensions of 200 μm×4 μm.

At 512, one or more material formation operations may be performed. For example, plasma focused ion beam (PFID) may be used to perform ion beam-induced deposition (IBID) may be performed to deposit a layer of material. The one or more material formation operations may include forming a material layer on exposed sidewalls of electrodes 420. The material layer may be a platinum layer or a gold layer. In some cases, the material layer may have a thickness of 200 μm. This material layer may cover a substantial area of 140 μm×50 μm to decrease surface impedance. In some cases, performing the one or more material formation operations may serve to prevent or reduce oxidation of the electrode sidewalls.

Embodiments provided herein include encapsulated microdevices that are substantially more resilient to deterioration. Bare microdevices, particularly when used in corrosive environments, can deteriorate rapidly and lose function. Bare microdevices can deteriorate in corrosive environments, while embodiments described herein of encapsulated microdevices can withstand such environments without decay for prolonged periods. Even with a thin, 100 nm thick layer of HfO2 and SiO2, the adverse effects of aging on a microdevice can be mitigated, thereby ensuring operational integrity and extended electronic component lifespan.

FIG. 6 illustrates impedance measurements for three electrodes, including: Al—140 μm×8 μm, Pt—140 μm×8 μm, and Pt—140 μm×50 μm. The Al electrode displays a high impedance (282 kΩ), primarily due to the presence of Al2O3. In contrast, despite possessing identical surface areas, the Pt electrode exhibits a significantly lower impedance, approximately 30 kΩ. Pt electrode measuring 140 μm×50 μm, demonstrates the lowest impedance of approximately 2 kΩ. By using low-impedance electrodes, the implant can stimulate effectively at smaller voltages (<3V).

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

1. A method of fabricating a device comprising:
processing a substrate by at least one of dicing or machining the substrate;
cleaning the substrate to remove debris;
assembling the device;
removing material to expose at least a portion of one or more electrodes of the device; and
forming a material layer on the at least the portion of the one or more electrodes of the device.

2. The method of claim 1, wherein forming the material layer on the at least a portion of the one or more electrodes comprises depositing a layer of platinum on the at least a portion of the one or more electrodes.

3. The method of claim 2, wherein depositing a layer of platinum on the at least a portion of the one or more electrodes comprises using plasma focused ion beam deposition (PFID) to perform ion beam-induced deposition (IBID).

4. The method of claim 1, wherein removing material to expose at least a portion of one or more electrodes of the device comprises performing one or more focused ion beam (FIB) milling operations on the device.

5. The method of claim 1, wherein the material removed comprises material in regions aligned with exposed portions of the one or more electrodes.

6. The method of claim 5, wherein the material removed comprises a dimension of around 200 μm×4 μm.

7. The method of claim 1, wherein cleaning the substrate comprises applying one or more solutions or liquids to remove the debris.

8. The method of claim 7, wherein the one or more solutions or liquids comprises one or more of deionized water or isopropyl alcohol.

9. The method of claim 1, wherein assembling the device comprises assembling components of the device using a silver epoxy.

10. The method of claim 9, wherein assembling the device further comprises encapsulating the device by forming a region that encapsulates the device.

11. A device, comprising:
a substrate comprising a first material, wherein a top surface of the substrate extends along a width dimension and a depth dimension;
a region comprising a second material formed in contact with the substrate, wherein a first sidewall of the region and a second sidewall of the region extend along a height dimension and the depth dimension, the first sidewall of the region parallel to the second sidewall of the region;
circuitry formed in the region comprising the second material;
a first electrode coupled with the circuitry, wherein a portion of the first sidewall of the region exposes a sidewall of the first electrode; and
a second electrode coupled with the circuitry, wherein a portion of the second sidewall of the region exposes a sidewall of the second electrode.

12. The device of claim 11, wherein the region comprises a coil, the coil coupled with the circuitry.

13. The device of claim 11, further comprising:
a first layer comprising a third material, the first layer formed in contact with the sidewall of the first electrode; and
a second layer comprising the third material, the second layer formed in contact with the sidewall of the second electrode.

14. The device of claim 13, further comprising:
a third layer comprising a fourth material, the third layer formed in contact with the first layer and the first sidewall of the region; and
a fourth layer comprising the fourth material, the fourth layer formed in contact with the second layer and the second sidewall of the region.

15. The device of claim 11, wherein a volume of the device is less than or equal to nine cubic microns.
